(19) United States
(12) Patent Application Publication
(10) Pub. No.: US 2005/0164411 A1
(43) Pub. Date: Jul. 28, 2005

(54) Quantitative protein assay using single affinity capture agent for identification and detection

(76) Inventor: Raymond A. Kim, Cupertino, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/010,056

(22) Filed: Dec. 9, 2004

Related U.S. Application Data
(60) Provisional application No. 60/527,838, filed on Dec. 9, 2003.

(51) Int. Cl. C12Q 1/68; G01N 33/543

(52) U.S. Cl. 436/518

(57) ABSTRACT

This invention discloses a single capture affinity assay and apparatus for the identification and quantification of an analyte in a biological sample. The assay uses an array of affinity capture agents specific for an analyte wherein the analyte is removed prior to detection. After the analyte is bound to the capture agent, a moiety of the complex is chemically modified, then the analyte is dissociated from the capture agent, revealing unmodified moiety located in the analyte-capture agent contact interface, which is detected by binding with a signal tag and the signal is quantitated. Analyte detection is achieved without using labeled secondary detection agents. The method is especially applicable to proteins, with antibodies as capture agents and selected amino acids as lysine being modified. The method may be used for uniplex or multiplexed analysis, and is applicable to high-throughput protein measurement.

### RELATED INFORMATION

This application claims benefit of and priority to U.S. provisional patent application Ser. No. 60/527,838 filed Dec. 09. 2003.

### FIELD OF INVENTION

The invention is directed to a method of identification and quantification of biomolecules, especially proteins, by a single capture agent assay technology. The invention is analyte specific, quantitative, and is applicable to antibody microarrays for uniplex or multiplexed high-throughput protein measurement.

### BACKGROUND OF THE INVENTION

Proteins are a major functional component of biological cells. The analysis of proteins is vital, both in basic biomedical research and in the biotechnology industry, especially in the discovery of new therapeutic and diagnostic entities. While accurate identification and quantitative measurement of small amounts of proteins has long been an endeavor in protein biochemistry, it has become even more important given the demands of proteomic research. The remarkable progress that has been made in genomic science using nucleic acid microarrays unfortunately has not been equally applicable to protein microarrays. Yet, the explosion of data from genomic research and the vast potential of its medical and biomedical industrial applications markedly increase the pressure for similar advances in proteomics research. Currently, therefore, there is a critical need in proteomics for sensitive methods for the accurate identification and detection of low abundance proteins in physiological mixtures.

It is well known that proteins are comprised of twenty naturally occurring L-amino acids, including neutral, hydrophobic and charged amino acids, which are linked by peptide bonds. Rarely, natural proteins may contain optical isomers, D-amino acids. Proteins may also be engineered to contain other chemical components, including synthetic amino acids and amino acid derivatives. Further, post-translational modifications of proteins, e.g., phosphorylation, are known to occur, many of which may be important for protein physiological function and regulation.

Amino acids on the surface of a protein are known to be involved in folding kinetics and maintenance of the structural stability of a protein. Surface accessible amino acids may also determine specificity of interactions of the protein with other proteins or other cellular components. The genetic sequence for the protein governs the linear sequence of the amino acids, which in turn influences the folding of the protein into its native structure by interactions of the charged and uncharged amino acid side chains with the surrounding microenvironment. Water soluble proteins have charged amino acids accessible on the surface, while hydrophobic amino acids are buried within the interior of the protein. This location strategy may be reversed, however, for lipid-soluble proteins, or for specifically maintained hydrophobic recognition sites. Protein denaturation occurs when the native structure of the protein is irreversibly changed, or is unable to return to a native state.

Various methods for the identification and quantification of proteins are known, including 2-D gel electrophoresis, mass spectrometry, sandwich-ELISA, bead assays, and antibody microarrays (for review, see Zhu H. et al., Annu Rev Biochem.2003; 72: 783-812). Immunoassay techniques such as sandwich-ELISA (enzyme-linked immunosorbant assay) have been widely used for measuring the expression levels of single proteins in biological samples. In sandwich-immunoassays, capture antibody is first immobilized onto a solid matrix (e.g. multi-well microplate, microscope slides), a query sample is added to effect analyte binding, followed by removal of unbound materials with buffer washes. The antibody-captured analytes are then detected by applying a secondary antibody with specificity for the same analyte, thus forming a sandwich complex. The secondary antibodies, also called the detection antibodies, are typically tagged with detection labels, and their signal intensities are measured for analyte quantification.

While useful for analysis of individual proteins, or uniplex analysis, many methods of protein detection are not easily amenable to the increased complexities of multi-analyte analysis. Multiplexed measurement of a mixture of low abundance proteins in natural fluids and tissues requires miniaturized, sensitive, and high-throughput technologies. Of the identification and quantification techniques currently used in proteomics research, antibody microarrays have technological potential for application to multiplexed experiments. Microarrays of antibodies offer the speed, scalability, and low volume capabilities required to support large scale protein profiling and quantitative analysis of complex biological samples.

Antibody microarrays are typically comprised of surface-immobilized antibodies arrayed in a manner similar to two-dimensionally spotted DNA microarrays. (Emil A Q, Nature Biotech. 2000; 18: 393-398; Dumoulin M., et al., Protein Sci. 2002; 11: 500-515; Kusnezow W., et al., Proteomics. 2003; 3:254-264). It is known that the immobilization of antibodies to solid matrix is possible because antibodies and their fragments (e.g. Fab′ and scFvs) have remarkably high structural stability and retain biological activity under diverse interfacial conditions. In addition, antibodies are relatively easy to produce from animals or by in vitro screening methods, e.g. phage display, thus facilitating content generation. Although monoclonal antibodies are often preferred for their higher specificity, both polyclonal and monoclonal antibodies are utilized in immunoassays, as well as antibody fragments, fusion fragments and antibody mimetic.

Currently, significant challenges exist in the development of antibody reagents for protein microarrays, especially ELISA immunoassays. One of the bottlenecks in the development of ELISA is the requirement of two validated antibodies that bind to an analyte without competing for the same binding site (or epitope). Microarray-based sandwich-ELISA requires two highly validated antibodies for each target analyte, one as a capture agent, the other for detection. The process of identifying antibodies that can work together as a pair in multiplexed sandwich-ELISA format, however, has proven to be an extremely challenging task when assembling large panels of microarray specificities (Haab B B, et al., Genome Biology. 2001; 2: 4-13). The difficulty lies with the technical and logistic nature of having to screen through many combinations of antibodies in order to match up a pair that binds to a cognate antigen with high affinity and specificity, but without competing for the same epitope.

A second difficulty in the development of ELISA is the selection of pairing status of antibodies as either capture or detection agents. Once paired, antibodies may need to undergo further optimization with regard to their relative orientation in the sandwich ELISA assay, as they may perform better as capture agents than as detection agents, and vice versa. Once configured, the antibodies then need to be screened again in the context of multiplexed assay for potential cross-reactivity amongst the specificities. These difficulties in antibody reagent development in ELISA are largely responsible for the extremely limited choices of multiplexable contents, which are currently limited to cytokines, chemokines, and growth factors.

In addition to the difficulties of antibody pair-matching constraint and orientation considerations, multi-analyte assays generally do not provide adequate detection sensitivity because of their inability to use signal amplification schemes commonly available to uniplex ELISA systems. The reason for this is because individual signals generated from enzyme-catalyzed amplification reactions, e.g. by horseradish peroxidase or alkaline phosphatase, diffuse and blend together rapidly in solution, thus losing the positional coordinate needed for analyte identification in an array. For this reason, the conventional protein microarray assays rely exclusively on the use of detection antibodies tagged with non-amplifiable label, e.g. fluorophores, phycoerythrin. The lack of signal amplification renders multi-analyte assays generally inadequate for applications requiring measurement of low abundance analytes.

Recently, investigators have proposed approaches for protein microarrays that relate to immobilization of a singles species of capture agent. US 2003/0153013 provides several approaches for antibody based array systems, one of which comprises a microarray of immobilized capture proteins onto a membrane. Detection is accomplished by the use of a hapten (such as biotin) attached directly to the analyte. Once the analyte-hapten is bound to the immobilized antibody, the complex is reacted with a detectable signal that recognizes the hapten., e.g., immobilized antibody binding a biotinylated analyte and reacted with a cyanine dye (such as Cy3) conjugated to streptavidin. US2004/0063124 describes a similar method for detecting a hapten-labeled analyte in a solution using a single species of capture agent on a solid surface. Again, a biotin and avidin system is described. Both of these methodologies, however, still utilize a sandwich-like technique for assay, in that for detection, a third component binds to the analyte while it is bound to the single capture agent. Both of these approaches also require the binding of a hapten to the analyte prior to affinity capture, which potentially may alter or interfere with native protein interactions for the analyte. Further, unbound hapten can be difficult to remove from the testing system, thereby increasing the background noise and decreasing detection sensitivity to low abundance proteins.

Despite technical difficulties, protein microarrays, especially arrays of antibodies, are a promising tool for proteomics research and biotechnological applications. A critical need exists for methodology for identification and quantification of peptides, polypeptides and proteins that is rapid and sensitive, and applicable to both uniplex and multiplexed analysis.

### SUMMARY OF INVENTION

This invention provides a method that permits identification and quantification of analyte using only one affinity agent, thus significantly simplifying the assay development process. This is achieved by using one affinity agent as both a capture agent to effect both specific binding to the target analyte (i.e. the protein identification) coupled with chemical modification steps, and to detect and quantify the binary binding between the capture agent and the analyte (i.e. the protein quantification). The requirement of a second detection agent is eliminated in this invention through the use of a selective chemical modification process.

The single affinity capture assay quantifies the area an analyte occupies in the capture agent by measuring the frequency of occurrence of a representative moiety that appears in the contact interface formed between the capture agent and the analyte upon their binding. The moieties located in the binding interface are generally shielded from the chemical modification reactions, which react only with solvent-accessible amino acid residues located elsewhere on the surfaces of the capture-analyte complex.

In this invention, the analyte is removed prior to detection and quantification. The removal of the analyte exposes the moieties located in the contact interface area. Through the use of a second selective chemical modification, these moieties in the interface regions can be specifically tagged and probed. The absolute quantification of the analyte in a query sample can therefore be determined by calibrating the signal obtained from a standard curve generated using known concentrations of the same analyte.

This invention may be applied to any biomolecule with an identified affinity agent and a target analyte for which a moiety exists that may be chemically modified under non-denaturing conditions.

This invention may be applied to the detection and distinguishing of various types of post-translation modification that are known to occur for proteins. For example, assay of phosphorylated proteins is greatly simplified by the use of only one antibody to quantify each phosphorylation state. This capability permits inclusion of a greater number of antibody specificities in multiplexed assay format for parallel measurement of multiple signal transduction pathways. This invention allows the mechanistic analysis of drug leads for target validation, potential off-target activities, the emergence of compensatory drug resistance mechanisms, and secondary or concurrent target assessment for combinatorial therapy.

This invention may be applied to comprehensive surveillance of pathogens and toxins, which is a critical component of bio-defense strategy. Over 50 biological agents including bacteria and spores (Gram positive and negative), viruses (DNA and RNA, enveloped and non-enveloped), protozoa, and toxins have been selected by the NIH as high priority select agents in order to spur therapeutic and diagnostic product development. Many of these select agents are also blood-borne pathogens transmissible by blood transfusions or tissue transplantations, a significant concern for the safety of our blood and organ supplies. Routine surveillance of these agents requires advanced screening technologies capable of assaying multiple pathogens and bio-toxins in a single sample. Currently, these tests are performed in uniplex assay format using immunological, serological, microbiological, or nucleic-acid based (i.e. PCR-based) methods. These tests, although suitable for single analyte measurement, are difficult to scale up for multi-analyte measurement, and even if feasible, the high cost of large scale assay production makes this approach impractical for routine screening. On the other hand, multiplexed assays using microarrays or beads can be developed for nucleic acids or proteins measurement, provided the chemical compositions of the analytes are compatible within the assay. For instance, RT PCR-based RNA detection has been multiplexed for diagnosis of viral pathogens, e.g. HIV and HCV. However, the difficulty of combining different PCR amplification strategies for different types of nucleic acids (for instance RNA and DNA molecules from a mixture of RNA and DNA viruses) makes large-scale PCR multiplexing technical challenging. In contrast, protein-based multiplexed assays, e.g. antibody microarrays, provide more versatile assay platform in that the antibodies specific for multiple types of pathogens (bacteria, viruses, and protozoa), bio-toxins (anthrax, botulism), and prions can be all multiplexed in a single assay for parallel detection. In addition to direct analyte measurement, the host's response to pathogens, e.g. protective antibodies, can also be profiled to detect the prior exposure or latent infection, thus adding additional dimension to the pathogen coverage. This invention provides an enabling technology with which to develop such multiplexed assays.

As applied to immunoassays, the present invention provides a method to measure the number of analytes bound to their cognate antibodies using amino acid modification chemistry as a means of differentiating antibody-analyte complexes from uncomplexed antibodies. The total number of analyte-antibody complexes formed in an assay system is proportional to the analyte concentration, and the calibration of this quantity against known concentrations of the analyte provides absolute quantification of the analyte in query samples.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention is not limited to require the details of the example embodiments.

### DESCRIPTION OF THE FIGURES

The details of the invention, including fabrication, structure and operation, may be gleaned in part by study of the accompanying figures, in which like reference numerals refer to like segments.

FIG. 1: A Flowchart of the Single Capture Agent Assay. The first step (Step A) involves immobilization of single capture agent onto a solid substrate. (Step B) Once the capture agent is immobilized, query sample containing the analyte of interest is applied to the microarray to allow formation of capture agent-analyte complexes. (Step C) The capture agent-analyte complexes are then subjected to the first selected chemical modification reaction under physiological conditions to block selected solvent-exposed residues on the exterior of the capture agent-analyte complexes. (Step D) The subsequent dissociation of the analyte from the immobilized capture agent exposes unmodified chemical residues located in the capture agent-analyte binding interface. (Step E) These unmodified chemical residues in the binding interface are then selectively subjected to a second chemical modification reaction with a heteroconjugate signal tag. (Step F) The covalent attachment of the signal tag to the unmodified chemical residues in the capture agent-analyte binding interface of the immobilized capture agent provides a non-diffusible, permanent signal, which may be measured quantitatively.

FIG. 2: The Schematic of Single Capture Affinity Agent Immunoassay. The first step (Step A) involves immobilization of capture agent antibodies onto a solid substrate. (Step B) Once the capture agent antibodies are immobilized, query sample containing the analyte of interest is applied to the microarray to allow formation of analyte-antibody complexes. (Step C) The antibody-analyte complexes are then subjected to the first amino acid chemical modification reaction under physiological conditions to block selected solvent-exposed amino acid residues on the exterior of the immunocomplexes. (Step D) The dissociation of the analyte from the immobilized antibodies subsequently exposes unmodified amino acid residues located in the antibody-analyte binding interface. (Step E) The amino acid residues in the binding interface are then subjected to a second chemical modification reaction, this time with a signal tagged heteroconjugate. (Step F) Once bound to the chemically blocked immobilized antibodies, the amount of bound signal tag can be measured, thus quantifying the amount of analyte bound at Step B.

FIG. 3: Graphical Presentation of Single Capture Affinity Assay Using Antibody and Chemically Modified Lysine. The first step (Step A) involves immobilization of capture agent antibodies (1) onto a solid substrate (2) to form an assay device (3). Amino acids, which are surface accessible (4), are exposed on the capture agent antibody (1). (Step B) Once the capture agent antibodies (1) are immobilized, query sample (5) containing the analyte of interest (6) is applied to the microarray (3) to allow formation of analyte-antibody complexes (7). (Step C) The antibody-analyte complexes (7) are then subjected to the first amino acid chemical modification reaction (8) under physiological conditions to block selected solvent-exposed amino acid residues on the exterior of the immunocomplexes (7). (Step D) The dissociation of the analyte (6) from the immobilized antibodies (1) subsequently exposes unmodified amino acid residues located in the antibody-analyte binding interface (4). (Step E) The amino acid residues in the binding interface (4) are then subjected to a second chemical modification reaction, this time with a signal tagged heteroconjugate (9). (Step F) Once bound to the chemically blocked immobilized antibodies, the amount of bound signal tag (9) can be measured, thus quantifying the amount of analyte (5) bound at step B.

FIG. 4: Detection of Specific Analyte. Dose titrations of specific and non-specific analytes were performed using the single capture affinity assay using a murine anti-human IgG Fc antibody as capture agent. Two-fold dilutions were made from 500 ng/mL to 15.25 ng/mL in PBS for both the affinity specific analyte, native human IgG Fc fragment, and the non-specific analyte, horse cytochrome C. The amount of absorbance (OD) of HRP-avidin signal tag bound to Fc fragment is shown as cross-hatched bars, while that of the non-specific analyte, horse cytochrome C, is shown as solid bars.

### DETAILED DESCRIPTION OF THE INVENTION

### DEFINITIONS

Affinity agent: As used herein, chemical moieties capable of binding and capture of a specific molecule. Also, a capture agent.

Amino acid: As used herein, is a chemical moiety that gives rise to a protein when polymerized into a polypeptide chain. Amino acids can include native L-amino acids, D-form amino acids, non-native amino acids and synthetic amino acids.

Analyte: As used herein, a molecule such as a polypeptide, whose identity, presence or quantitative amount is to be determined. The “analyte” may include peptides, proteins, enzymes, receptors, hormones, transcription factors, viral proteins, bacterial proteins, glycoproteins, carbohydrates, lipids, lipid proteins, nucleic acids, small molecules and compounds, therapeutic chemicals such as antibiotics, interleukins, acute phase response proteins. Analytes may be of plant, animal, viral or bacterial origin.

Antibody: As used herein, a polypeptide substantially encoded by an immunoglobulin gene or immunoglobulin genes, or fragments thereof, which specifically bind and recognize an immunogen. The recognized immunoglobulin genes include the kappa, lambda, alpha, gamma, delta, epsilon and mu constant region genes, as well as the plurality of immunoglobulin variable region genes. Antibodies exist, e.g., as intact immunoglobulins or as a number of well characterized fragments produced by digestion with various peptidases. This includes, e.g., Fab′ and F(ab)′.sub.2 fragments. The term “antibody,” as used herein, also includes antibody fragments either produced by the modification of whole antibodies or those synthesized de novo using recombinant DNA methodologies.

Antibody affinity: As used herein, an antibody “is specific,” has “affinity for” or “specifically binds” to a protein when the antibody functions in a binding reaction which is determinative of the presence of the protein in the presence of a heterogeneous population of proteins and other biological entities. Thus, under designated immunoassay conditions, the specified antibodies bind preferentially to a particular protein, and do not bind in a significant amount to other proteins present in the biological sample. Specific binding to a protein under such conditions requires an antibody that is selected for its specificity for a particular protein.

Biological sample: As used here, is biological fluids, extracts, cells, tissues. Biological samples may include, but are not limited to, blood, serum, plasma, cerebrospinal fluid, lymphatic fluid, semen, urine, sputum, synovial fluid, lacrimal tears, saliva, nipple aspirate, eye fluid. Biological samples can be epidermal, mesodermal or endodermal cells or extracts, or any combination thereof, from biological tissue, organ and/or cell culture. Biological samples may be of animal, plant, bacterial; viral or prion origin.

Biomolecule: As used herein, polypeptides, polynucleotides and other polymers that display specific affinity toward a corresponding binding partner.

Blocking: As used herein, the process of chemically modifying amino acids of affinity capture agent which are not directly involved in analyte binding.

Capture agent: As used herein, biomolecule capable of specifically binding to an analyte. Examples of a “capture agent” include an antibody, monoclonal antibody, polyclonal antibody, antibody fragments, antibody peptides, antibody mimetics, antibody fusion proteins, phage display, nucleic acid aptamers, fibronectin display, peptide-nucleic acid aptamers, non-antibody protein scaffolds.

Chemical modification: As used herein, derivatization of a moiety, such as an amino acid, by a reagent specific for the moiety.

Dissociation: As used herein, the process of removing bound analyte from affinity capture agent, may be termed a stripping agent.

Immunoassay: As used herein, an assay in which the capture or detection agent is derived from an immunoglobulin gene, which may be an antibody, antibody fragment, or a derivative or analogue thereof.

Microplate: As used herein, support composed of glass, plastic, polystyrene or polypropylene containing a number of wells wherein capture agent is immobilized. An industry standard is the 96 well microplate.

Microarray: As used herein, miniaturized device containing multiple number of immobilized probe elements, an ordered matrix of discretely placed capture agents on a support. A linear or two-dimensional array of preferably discrete regions, each having a finite area, of species of capture agents formed on the surface of a support. This may also be referred to as a protein array, protein chip or protein biochip.

Multiplexed assay: As used herein, an assay measuring more than one analyte in parallel from a single sample. In multiplexed assay, there is more than one analyte, and many different analytes may be queried simlultaneously.

Non-denaturing: As used herein, a reaction condition which does not cause structural damage to biomolecules.

Post-translational modification: As used herein, a process that modifies primary polypeptides by addition or deltion of components. This includes chemical modifications, including but not limited to phosphorylation, glycosylation, acetylation, and deletion of components, including but not limited to proteolysis or intein modification.

Protein or polypeptide: As used herein, biomolecules comprised of multiple number of amino acids, a polymer composed of amino acid residues, related naturally occurring structural variants, and synthetic non-naturally occurring analogs thereof linked via peptide bonds, related naturally occurring structural variants, and synthetic non-naturally occurring analogs thereof.

Signal amplification: As used herein, a condition where more than a 1: 1 linear relationship detection is incurred.

Signal intensity: As used herein, an indication of signal tag binding, measurement and subsequent quantitation of bound signal tag.

Sandwich ELISA: As used herein, an immunoassay in which both a capture antibody and a detection antibody bind to a targeted analyte.

Signal tag: As used herein, a molecule with a physical property that is analyzable by a detector. A signal tag may include a dye that is fluorescent, chemiluminescent, light-scattering, nano-crystalline, calorimetric, or radioactive, or any combination thereof.

Substrate: As used herein, a material to which biomolecules are physically attached. For microarray, a substrate can be planar materials such as slides, microwells, silicon wafers, membrane, hydrogel, or can be 3-dimensional materials such as beads, capillaries.

Uniplex assay: As used herein, an assay measuring a single analyte.

Washing: As used herein, the process of removing unbound materials from assay using buffer solutions.

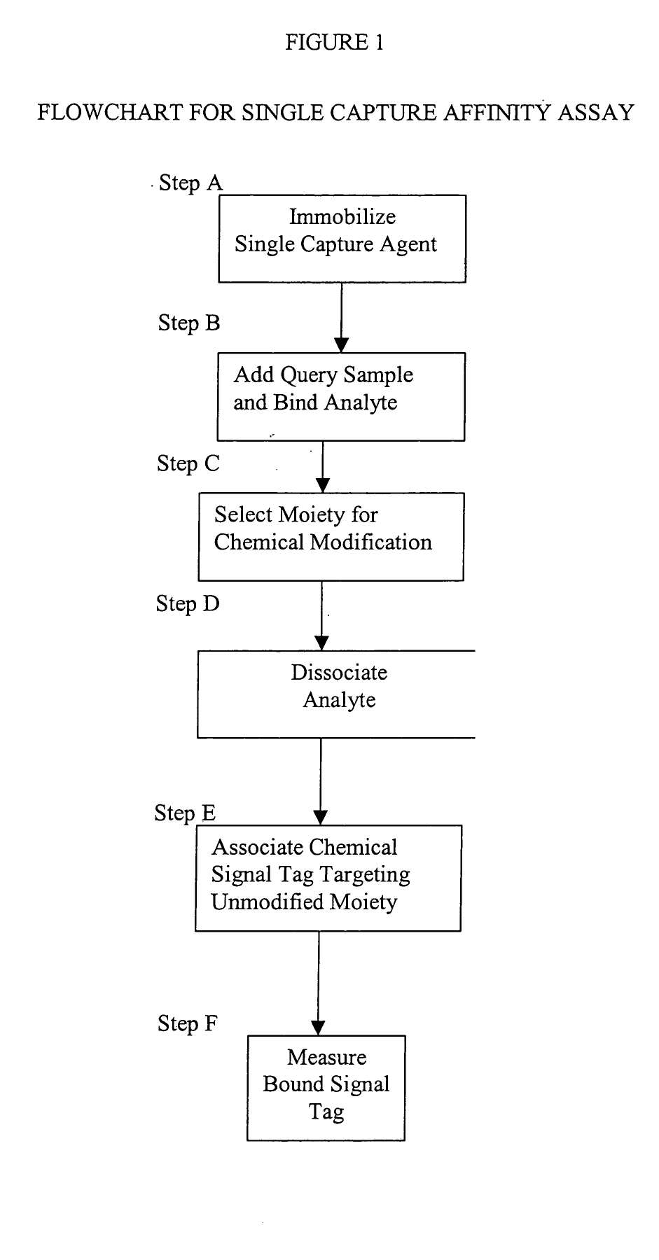

Outline of the Single Capture Affinity Assay. A flowchart of the Single Capture Affinity Assay as generally applied to biomolecules is shown in FIG. 1. The method is described in detail below for the use of antibody as a capture agent, but other biomolecules capable of specifically binding to analyte with high affinity, e.g. nucleic acid aptamers, peptide-nucleic acid aptamers, non-antibody protein scaffolds, can be used equally well. Also, the method is provided for lysine modification, but this method is broadly applicable with any-other amino acids for which there are specific chemical modifiers available.

In Step A, an array of a single capture agent with a known affinity for the targeted analyte is immobilized on a solid substrate. The query sample containing the targeted analyte is then added in Step B and allowed to react with the immobilized single capture agent. Two sequential chemical modifications are then performed, which differentiate the chemical moieties that are in direct contact with the analyte from the ones that are not involved in analyte binding. A moiety is selected for the first chemical modification for Step C. This is the “blocking” step that takes place after affinity capture agent-analyte complexes have been formed in Step B. This blocking modification is performed under physiological conditions to target all such solvent-accessible moieties on the capture agent-analyte complex. Any number of moieties, both natural and synthetic, can be targeted in step three, provided they meet certain criteria: (1) the moieties must be on the surface of the protein; (2) the moieties must be amenable to chemical modification, and (3) the chemical modification must occur under non-denaturing conditions to maintain the native tertiary and quaternary structure of the single capture agent. In Step D, the analyte is disassociated from the single capture agent. Then, in Step E, the single capture agent is reacted with a second chemical modification, which specifically recognizes the unmodified moiety. This second chemical modification associates the unmodified moiety with a signal tag. The amount of bound signal tag is then measured in Step F. Alternatively, amplification of the signal tag prior to measurement increases detection sensitivity.

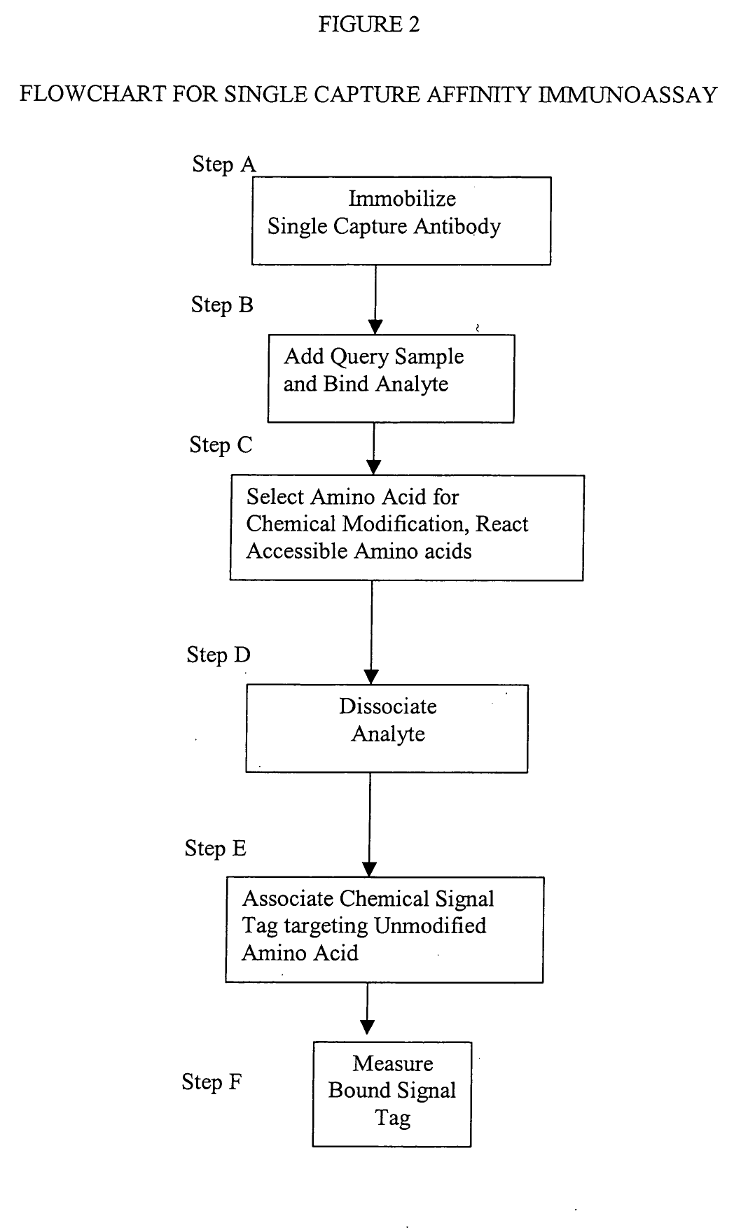

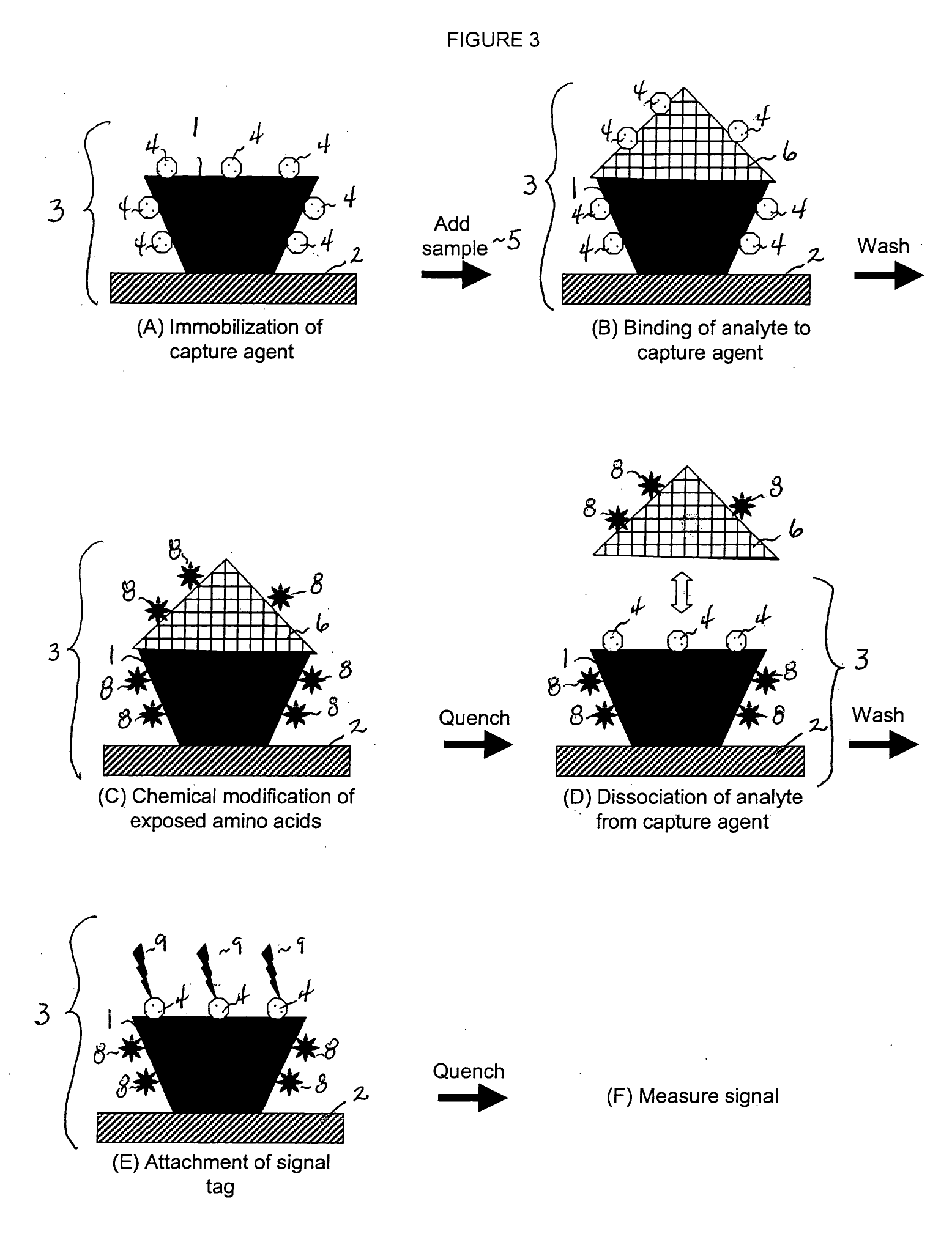

Outline of the Single Capture Affinity Assay for Immunoassay. A flowchart of the Single Capture Affinity Assay as applied to immunoassays is shown in FIG. 2, and is presented graphically in FIG. 3.

Step A: A capture antibody (1) is first immobilized onto a solid surface (2). The immobilization can be a covalent bonding via chemical or photo-activated cross-linkers, or non-covalent bonding using, for example, biotin-streptavidin interaction. The solid phase where the antibody immobilization takes place can be of any number of different types including such substrates as glass slides coated with appropriate materials, wells of microplates, or even semi-conductor materials, e.g. silicon wafer.

In terms of the type of antibody (1), this invention is compatible with both monoclonal and polyclonal antibody types, as well as antibody fragments, antibody fusion proteins and peptides. Polyclonal antibodies, even with affinity purification, tend to give higher non-specificity, and for this reason, it is preferable to use monoclonal antibodies as capture agent antibody.

The placement of capture antibody (1) onto a solid surface (2), the process called spotting, can be performed using such methods as manual spotting with pointed tip, manual or robotic contact spotting with pin tool, or by non-contact ink-jet method.

A unit of solid surface containing immobilized capture antibody is referred to herein as the “assay device” (3). The assay device (3) can contain single or multiple types of capture agents (1) specific for one analyte (6). The assay device can also contain single or multiple types of capture agents, each specific for different analytes, to allow simultaneous measurement of many analytes in a multiplexed format. This invention allows identification and quantification of analytes in a number of different assay formats such as measuring single analyte per assay; (i) solid phase assays measuring single analyte per assay, and (ii) solid phase assays simultaneously measuring plurality of analytes per assay, for example, in a microarray chip device.

Step B: Once immobilized, the capture antibody (1) is incubated with a sample (5) containing analyte of interest (6), which binds with affinity and specificity to the capture antibody (1). Binding of analyte (6) is allowed to occur such that complexes of antibody-analyte (7) are formed, typically 1 hr at room temperature for immunoassays. The analytes (6) can be presented to the capture antibody (1) singly or in a reconstituted mixture with other biomolecules, as would be the case for calibration assays. Alternatively, the analyte (6) can be presented as a natural constituent of complex biological matrixes, e.g. serum, plasma, urine, lacrimal fluid, synovial fluid, cerebrospinal fluid, or a cell or tissue lysate. After a suitable incubation time, unbound materials are removed from the assay device by flushing with appropriate buffer solution.

In the following steps two sequential amino acid modifications are performed to differentiate the amino acid residues of antibody which are in direct contact with the analyte from those which are not involved in analyte binding.

Step C: The first modification reaction, termed “the blocking step”, takes place after analyte-antibody complexes (6) (i.e. immunocomplex formation) have been formed. This blocking modification is performed under physiological conditions to target solvent-accessible amino acids (4), which are generally located on the surfaces of the antibody-analyte complex, for chemically modification (8). Note that amino acids (4) which happen to be in the binding area interface of the immunocomplex (6) are not exposed to this chemical modification (8).

Any number of amino acids, both natural and synthetic, can be targeted in this step, provided they meet certain criteria: (i) they must be on the surface of the protein; (ii) they must be amenable to chemical modification, and (iii) the chemical modification occurs under non-denaturing conditions, that is where the native tertiary and quaternary structure is maintained. Chemical modifiers which can specifically and covalently modify a target amino acid must be available, and such chemical modifications must be made under non-denaturing conditions in order to preserve the conformational integrity of the proteins.

Amino acids such as lysine, histidine, tyrosine, arginine, glutamate, aspartate, tryptophan, cysteine, and methionine can generally satisfy the above criteria. For examples, lysine can be modified by N-hydroxysuccinimide or citraconylate (Kvaratskhelia et al., Proc. Natl. Acad. Sci. USA, 2002: 99:15988-93), methionine by iodoacetamide (Falkenstein et al, Mol. Cell Biochem. 2001; 218:71-9), cysteine by maleimide (Lundblad, Techniques in Protein Modification, CRC Press, Boca Raton, Fla., USA, 1994, pp. 91-96), tyrosine by acetylimidazole or tetranitromethane (Beckingham et al, Biochemical J. 2001; 353:395-401), arginine by phenylglyoxal or cyclohexanedione (Degenhardt et al, Chem. Mol. Biol. 1998; 44:1139-45), glutamates and aspartate by Woodwards reagent K (Bahar et al, Amer. J. Physiol. 1999; 277:791-9), histidine by diethylpyrocarbonate or 4-hydroxy-2-nonenal (Kalkum et al, Bioconjugate Chemi8stry, 1998; 9:226-35), and tryptophan by N-bromosuccinimide or 2-hydroxy-5-nitrobenzyl bromide (Xue et al, Biochem. Cell Biol. 1997; 75:709-15).

Lysine is a preferred amino acid for chemical modification in this invention, because the chemistries around the modification of lysine side chains are well-characterized and have been extensively practiced in various aspects of immunochemistry and protein structural studies. This, together with a large number of commercially available and customizable reagents, make lysine a preferable choice of amino acid for the single capture agent assay.

The most commonly used lysine-specific covalent modifier is N-hydroxysuccinimide (NHS). This and various ester conjugates of NHS were tested for their activity in the assay. One of the most salient properties of NHS is the feasibility of performing modification reactions under physiological conditions, such that selective modification of solvent-accessible lysine residues can be achieved without perturbing the overall protein structure (Hanai et al, Proc. Natl. Acad. Sci. USA, 1994; 91:11904-8). The chemical modification of immunocomplexes with NHS followed well-validated, published protocols (Id.).

The lysine modification reactions will be optimized using NHS esters containing fluorophore. NHS conjugates of fluorescein, rhodamine, courmarine, oxazine, or carbopyronin are readily available from many vendor (Kinoshita et al, Nuc. Acids Res. 1997; 25:3747-8). By tracking the fluorescence from NHS as a reporter, the kinetics and the efficiency of cross-linking can be readily monitored for assay optimization. Quenching of NHS at the end of the reaction will be achieved with Tris buffer containing L-lysine (Pütz et al, Nuc. Acids Res. 1997; 25:1862-63).

The chemical modification of the selected amino acid (4), e.g., lysine, in Step C takes place under non-denaturing conditions such that only the native lysine residues (4) located on the solvent-accessible surfaces of the capture-analyte complex (7) are selectively modified (8). The extent of chemical modification should be near stoichiometric such that the majority of solvent-accessible lysines are modified.

The reaction conditions for this step are tailored to known conditions such that the lysine modification avoids denaturing or otherwise adversely affecting the functionality of the antibodies. Conditions were also tailored such that analytes cannot dissociate from the capture antibodies during the chemical modification reaction. For the practice of the invention, the affinity of antibody (i.e. K_{D}) is selected to be high enough to form and maintain a stable immunocomplex, and thereby to avoid the preemptive blocking of lysine residues in the analyte binding domain of antibody. Where the specific affinity is ot known in advance, it is well within the ordinary skill of those in the art to verify antibody analyte affinity experimentation.

In another embodiment, the chemical modifier can be a heterobifunctional cross-linker with one end carrying a molecular tag, e.g. fluorophore, while the other end carries a lysine-reactive moiety, e.g. N-hydroxysuccinimide. Use of such heterobifunctional reagent results in fluorescent labeling of the capture antibody whose signal, once the bound analyte is removed, corresponds to the amount of spotted capture antibody. This information can be used to normalize and correct for any variability arising from spot-to-spot inconsistency in antibody spotting.

The chemical modification in Step C is terminated by the addition of large volume of amine-containing reagents, e.g. Tris buffer, followed by several washes with phosphate-buffered saline. In this way, surface accessible amino acid residues (4) modified in this manner are blocked (8) and prevented from participating in subsequent amino acid modification,

Step D: Upon completion of the chemical modification of the selected amino acid (3), e.g., lysine, the assay device (3) is then treated with a reagent e.g., low pH solution, to dissociate the antibody-analyte complex (7). Dissociation of the analyte (6) from the capture agent (1) exposes unreacted amino acids (4) which escaped the first chemical modification (8) (in Step C) by virtue of being buried in the interface formed by binding of the analyte to the capture antibody. Such contact interface generally occupies a large area and excludes most molecules due to their tight binding nature. Other reagents, besides low pH solution, can be used to force the dissociation of analyte from the capture. The conditions are selected to avoid disruption of the native structure of capture antibody, and to avoid detachment from the solid phase substrate.

The dissociated analyte is now removed from the assay device. Note that unreacted amino acids (4) may also be present on the binding area of the analyte (6). The assay device (3) is washed to remove all dissociated analyte. Solvent exclusion from contact interfaces of protein-protein, antibody-protein, and protein-nucleic acids complexes has been amply demonstrated in the literature.

The antigen dissociation reagents used for the treatment of the immunocomplexes are selected to avoid irreparable damage to the antibodies. However, because the heavy and light chains of IgG antibody molecules are typically held together by 16 intra- and inter-chain disulfide bonds, partial unfolding of antibodies during dissociation can rapidly be reversed by washing the microarrays with a neutralizing buffer, e.g. PBS. The treatment of antibodies with ImmunoPure® (Pierce Biotechologies, Inc., #21004) has not been observed to cause any detectable loss of their binding activity.

Step E: The newly exposed amino acid residues (4) located in the binding interface of the capture antibody are now targeted for a second round of chemical modification with a signal tag (9). In the preferred case of lysine, modification is performed using a heterobifunctional cross-linker with a lysine-reactive group (e.g. N-hydroxysuccinimide) at one end and a molecular tag at the other end. The molecular tag can be any number of fluorophores, e.g. Cy3, Cy5, fluorescein, or enzymes, e.g. horse radish peroxidase or alkaline phosphatase which can generate fluorescent, chemiluminescent, or spectrometric signal through its catalytic activity.

Step F: The intensity of signal generated from the polymerized nucleotides is now measured. The intensity correlates with the number of signal tag (9)-conjugated lysine residues, and therefore they indicate the number of lysine residues which are located in the capture-analyte contact interface. The total quantity of the signal tag (9)-conjugated lysine residues in the assay device therefore directly correlates with the total amount of space occupied by the analytes on the capture agent (1), which is in turn governed by the quantity (or concentration) of the analyte (6). Therefore, the absolute concentration of the analyte (6) in any given sample can be determined by correlating the signal generated from the sample to a standard curve generated by multi-point calibration assays with the known concentrations of the same analyte.

The assay validation covered the linearity, sensitivity,. reproducibility, matrix effects, and cross-reactivity with specific target range (Deshphande S S. Enzyme Immunoassays from Concept to Product Development, Chapman & Hall, N.Y., 1996).

Linear Dynamic Range: Linear range of the assay refers to the concentration range of target analyte that can be confidently measured. Dynamic assay range on the protein assay device is specific for each antibody and in general at least 3-logs of concentration should be accurately measured. Dose response testing is performed by dilution of antigens to cover 4-5 logs of concentration range. The resulting calibration data is log-transformed and graphed as signal intensity vs. analyte concentration. Data corresponding to the linear range of the resulting plot is fitted to a regression to determine the linear range of assay response. The slope of the resulting line indicates the concentration-dependent signal response over the tested concentration range.

Sensitivity: The lower end of the dynamic linear range represents the lower limit of detection (LOD). LOD is measured at a predetermined % CV (coefficient of variation) where CV is the ratio of standard deviation and signal intensity. The Z-factor is used to determine the significance of signal typically at 95% confidence (Zhang et al, J. Biomol, Screen, 1999; 4:67-74), i.e. 2 standard deviations from the background.

Reproducibility: Reproducibility analysis measures the variance of signal from the same sample applied in two independent biochips, or in different wells of array-of-array. The reproducibility is measured at the most robust ranges of each.

Matrix effect: Matrix testing is performed to verify that the response and accuracy of the assay is compatible with a given biological sample matrix. To test this parameter, a cocktail of purified proteins is added to the undiluted biological matrix (e.g. stripped serum, culture medium, cell lysate, etc) at a known concentration, and the resulting test sample assayed using the protein assay device (3). Multiple test samples are tested, each containing different concentrations of each analyte. To correct for endogenous levels of protein measured in the matrix, unspiked matrix samples are assayed as a baseline control. The resulting data are compared to standard curves generated in a buffer diluent. Analyte concentrations in the test sample are quantified by direct interpolation from this standard curve, and compared to the known quantities present in the test sample.

Cross-reactivity: Cross-reactivity analysis is typically performed by systematically removing one analyte at a time from the multiplexed mixture of analytes, while the rest of the multiplexed is kept at concentrations where strong dose-response behaviors are obtained. The cross-reactivity is then calculated by the ratio of the drop-out signal to the signal of the analyte of interest when present in a control.

### EXAMPLE 1

Antibody Immobilization (Corresponding to Step A of FIGS. 1-3). A murine IgG monoclonal antibody (US Biologicals; Swampscott, Mass.; # 11903-60) specific for human IgG Fc fragment was immobilized onto a solid support, the bottom of 96-well microplates by incubating 50 μl of 100 ng/ml antibody in phosphate-buffered saline (PBS; USB Corp., Cleveland, Ohio; #75889) for 1 hr. A consideration was the selection of microplate substrate compatible with the single affinity capture assay. Several microplate types, e.g. treated, untreated, and coated polystyrene as well as polypropylene, were tested. The results showed that different types of plastics performed differently in the assay, and even the same type of plastic from different vendors gave widely varying performance. The factors considered in the choice of solid support included capability of protein binding and amount of inherent background signal. Of the solid supports tested, the γ-irradiated polystyrene plates (E&K Scientific Products, Campbell, Calif.; #26101) gave the most reliable assay performance (data not shown) and were chosen as the microplate substrate for the single capture affinity assay development.

The wells with the immobilized capture antibody were washed and treated with a blocker, such as 2% polyvinylpyrrolidone, prior to use to prevent spurious binding of biomolecules in the assay. Protein-based blockers, e.g. bovine serum albumin (BSA), gelatin, non-fat milk powder, are commonly used in microplate immunoassays. However, since the invention of the single capture affinity assay relies on specific modification of selected amino acid residues, protein blockers can also become substrates for these chemical modifications, and therefore could interfere with the assay performance by increasing high background noise. For this reason, it is preferably to use only non-proteinaceous blockers or peptide mixtures not containing the selected amino acid of interest. Preferably, blocking with 2% polyvinylpyrrolidone (PVP; Boston Bioproducts; Worchester, Mass.; IBB-#190) for 1 hr at room temperature produced consistent well blocking without interfering with the amino acid modification (data not shown).

Antigen Binding and Chemical Modification of Solvent-exposed Lysine Residues (Corresponding to Steps B & C of FIGS. 1-3). The functionality of the immobilized antibody was confirmed using biotinylated human Fc fragment (Bethyl, Inc., Montgomery, Tex.; #P80-104) as the antigen. The Fc fragment was biotinylated as per published protocols (Mendoza et al, Biotechniques, 1999; 27:778-80). The binding reaction was performed by incubating 50 μl of the analyte in PBS-20% glycerol for 1 hr at room temperature. After rinsing out unbound analyte, HRP-avidin (Zymed Laboratories, Inc., S. San Francisco, Calif.; #43-4423) was added to bind the antibody-captured biotinylated antigen. After 1 hr of incubation, unbound HRP-avidin was washed away and a HRP substrate (Pierce Biotechnology, Rockford, Ill.; #37615) was added to develop the colored signal. A titration assay with biotinylated analyte showed linear dose response from 1-500 ng/ml range (data not shown), thus confirming that the antibody was immobilized and retained functionality.

An amine-reactive modifier N-hydroxysuccinimide (NHS) (EMD Biosciences, San Diego, Calif.; NHS #01-62-0009) was used to modify solvent-exposed lysine residues of the immunocomplexes. An optimal concentration was determined to find the correct dosage of NHS to bring about comprehensive blockage of lysine residues without damaging the functional integrity of the antibody. A titration of NHS was performed to identify the optimal window of NHS concentration. For this, a 50 μl aliquot of 50 ng/ml biotinylated analyte was first allowed to bind to the immobilized antibody, and the resulting immunocomplexes were then treated with 50 μl of NHS solutions covering 0 to 4% (w/v) concentration range. After 30 min at room temperature, NHS solutions were aspirated and 100 μl of 0.1 M Tris (pH 7.4) containing 0.1 M lysine was added to quench the residual NHS.

TABLE 1
The concentration at which N-hydroxysuccinimide
destroys antibody function was assessed by
measuring the fraction of antibody-bound analyte
at several different concentrations of NHS. Up
to 2% NHS was found to be safe for antibody activity.

| NHS (%) | % Analyte Bound |
|---|---|
| 0 | 100 |
| 0.25 | 99.2 |
| 0.5 | 95.3 |
| 1 | 93 |
| 2 | 89.7 |
| 4 | 40.1 |

Table 1: Optimization of NHS treatment. A range of NHS concentration was tested to determine the concentration at which NHS causes inactivation of antibody function as indicated by loss of antigen-binding activity. The length of NHS treatment time from 0-60 min has been examined using 2% NHS. At each time point, NHS was removed and quenched with Tris-Lysine buffer, and the amount of antibody-bound antigen was measured as described in the text.

The results (Table I) showed that when compared to untreated sample (0%), the wells treated with 0.25%, 0.5%, 1%, and 2% NHS retained most of the captured analyte, indicating that up to 2% NHS did not cause inactivation of the immobilized antibodies. In contrast, treatment with 4% NHS caused greater than 60% reduction in the amount of retained analyte relative to untreated sample, indicating significant inactivation of the antibody. All values shown were averages of duplicate data points.

In Table 2, the lenght of NHS treatment time from 10 to 60 min was examined using the same assay design as in Table 1. At 2% NHS concentration, there was negligible loss of antibody function up to 30-45 min of treatment. By 60 min of incubation, approximately 5% of the capture analyte was lost when compared to untreated samples. Based on these results, 2% NHS for 30 min at room temperature was chosen as the standard condition for all subsequent NHS treatment.

TABLE 2
The length of treatment time with N-
hydroxysuccinimide was tested to evaluate the safety of NHS
treatment on antibodies by as. Up to 60 min of treatment time
with 1% N-hydroxysuccinimide at room temperature was shown
to be safe for antibodies as demonstrated by retention of 94.5%
of the bound analyate.

| Incubation Time (min) | % Analyte Bound |
|---|---|
| 0 | 100 |
| 10 | 99.4 |
| 20 | 99.8 |
| 30 | 98.2 |
| 45 | 97.3 |
| 60 | 94.5 |
| No Analyate | 2.8 |

Table 3: Optimization of analyte dissociation from immobilized antibody. A commercially available antibody disassociation agent was evaluated at three different concentrations. As shown in Table 3, ImmunoPure® IgG Elution Buffer (Pierce Biotechnology, Inc., Rockford Ill.; #21004) was found to be significantly effective in stripping bound antigens from antibodies. The length of time required to strip antibody using 1× ImmunoPure® was examined. Complete dissociation required at least 30 min of incubation. The effect of ImmunoPure® on the antigen-binding function of the antibody has been examined. Comparison of antibodies which had been pre-treated with ImmunoPure® for 30 min to that which had not been treated showed that ImmunoPure® did not damage immobilized antibodies and was safe to use in the assay. The low background values indicated the background signal was produced without the antigen being present.

TABLE 3
The concentration of dissociator needed to remove
antibody-bound analyte was determined. The treatment
of antibody-analyte complex with full strength (1X)
ImmunoPure ® removed 97.7% of the pre-
bound analyte from the analyte. Dilutions of
ImmunoPure ® by 2- and 4-fold removed
95.1% and 92.2% of analyte, respectively.

| Dissociator Conc | % Analyte Bound |
|---|---|
| 1X | 2.3 |
| 0.5X | 4.9 |
| 0.25X | 7.8 |

Analyte Dissociation (Corresponding to Step D of FIGS. 1-3). Subsequent cross-linking of signal tag to the lysine residues in binding area first required dissociation of captured antigen from the antibody. Any number of dissociation conditions can be used to remove the analyte from the capture agent, as long as such conditions do not substantially denature the single capture agent. Available options range from relatively harsh conditions, e.g. low pH, potassium thiocyanate, urea or guanidine treatment ), to more gentle conditions, e.g. dioxane or ethylene glycol at neutral pH (Summaria L, et al. J. Biol. Chem. 1970; 246:2136-42; Gennaro W D, et al. Clin. Chem. 1975; 21: 873-879). In this invention, however, it is necessary to safeguard the structural integrity of the single capture agent, e.g., antibodies. The use of harsher treatments, e.g. protein denaturants, detergents, and extreme pH conditions, are avoided. Antibody dissociation agents, also called stripping reagents, are also commercially available. For certainty, the suitability of any reagents or conditions can be verified by testing the retention parameters in a small scale test.

A commercially available antibody stripping reagent, (ImmunoPure® (Pierce Biotechnology, Rockford, Ill., #21004), was tested in the single capture affinity immunoassay. The chemical compositions of this reagents is proprietary and undisclosed, but such reagents have been formulated to produce antigen dissociation from antibody affinity columns without causing damage to antibodies. As shown in Table 3, ImmunoPure® effected dissociation of the biotinylated Fc fragment from the antibody in a concentration-dependent manner. ImmunoPure®, even at 0.25× strength, demonstrated substantial antigen dissociation from the antibody capture agent.

TABLE 4
The length of time required to completely dissociate analyte
from antibody was determined. Using the full strength of
ImmunoPure ® at room temperature, approximately
30-45 min was needed to completely remove the analyte
to background level.

| Incubation Time (min) | % Analyte Bound |
|---|---|
| 0 | 100 |
| 10 | 15.5 |
| 20 | 8.9 |
| 30 | 4.2 |
| 45 | 3.6 |
| 60 | 3.1 |

The length of treatment time with the full-strength (1×) ImmunoPure® was also examined. As can be seen in Table 4, analyte stripping was time dependent and required approximately 30 min of incubation at room temperature with under static conditions (no shaking). Incubations shorter than 30 min produced incomplete dissociation.

To determine whether the loss of captured antigen was due to antigen stripping or to inactivation of antibody by ImmunoPure®, an experiment was conducted where immobilized antibody was pre-treated with ImmunoPure® prior to antigen binding. By comparing to untreated antibodies, inactivation of antibody by ImmunoPure® can be readily differentiated from antigen stripping. As shown in Table 5, pre-treatment of immobilized antibodies with ImmunoPure® did not diminish the ability of the antibody to bind the analyte when compared to the antibodies which were not pre-treated with ImmunoPure®. This experiment clearly demonstrated that ImmunoPure® was safe to use, and was effective in dissociating the captured antigens from the antibody. Based on these results, the analyte dissociation using 1× ImmunoPure® for 30 min was chosen as the standard stripping condition.

### TABLE 5

TABLE 5
The effect of dissociation reagent on the antibody
was tested by treating antibody with the reagent prior to analyte
binding. The antibodies pre-treated with the dissociation
reagent retained full antibody function when compared to
untreated antibody.

| Condition | % Analyte Bound |
|---|---|
| Untreated | 100 |
| Pre-treated | 98.9 |

Cross-linking of signal tag to single capture affinity lysine residue and signal development (Corresponding to Steps D and E of FIGS. 1-3). The single capture affinity assay of the invention may be performed using a uniplex assay system using a biotin-conjugate of NHS (NHS-biotin; Pierce Biotechnology, Rockford, Ill.; #21329) as a signal tag. The reaction chemistry of NHS-biotin is identical to that of NHS, and therefore provides a much simplified alternative to tagging the lysine residues in the single capture affinity assay. With NHS-biotin cross-linked to lysine residues, the signal development can be readily allowed by biotin-specific binding of HRP-avidin, followed by catalysis of an exogenously supplied HRP substrate. The rate of catalysis of HRP substrate can then be determined as a measure of cross-linked lysines.

In Table 6, the results of the invention assay for immunoassay are presented. The single capture affinity assay was performed according to the steps as outlined in FIGS. 1-3. A murine anti-human IgG Fc antibody was used as a capture agent, and 2% polyvinylpyrrolidone was used to block the assay wells. The analyte (the native human IgG Fc fragment) was titrated into individual wells to determine the dose responsiveness of the assay. Horse cytochrome C (USB Corp, Cleveland, Ohio # 14102) was also tested in parallel in separate wells as a control for non-cognate antigen binding. Unbound antigens were removed by washing, and 2% NHS was added to block solvent-exposed lysine residues. After quenching with Tris-lysine, the captured analytes were stripped using 1× ImmunoPure® for 30 min, and the newly exposed lysines in the single capture affinity space were cross-linked with 0.003125% (w/v) NHS-biotin. This particular concentration of NHS-biotin was determined from a preliminary experiment (data not shown). After 30 min of treatment, NHS-biotin was quenched and 50 μl of 1:4000 dilution of HRP-avidin (Zymed Laboratories, Inc., S. San Francisco, Calif.; # 443-4423) was added for 1 hr. Biotin-bound HRP-avidin was then quantified by monitoring the absorbance (optical density, OD) of the colored product developed from the catalysis of a HRP substrate as per manufacture's recommended protocol. After an incubation at room temperature for 15-30 minutes, the absorbance at 416 nm was measured by a microtiter plate EIA reader.

TABLE 6
Dose titrations of specific and non-specific analytes were
performed. Two-fold dilutions of analyte were made from
500 ng/mL to 15.625 ng/mL in PBS. The absorbance (OD)
at 416 nm was measured for both the affinity analyte, the Fc
fragment, and the non-specific analyte. The assay showed
specific detection of Fc analyte in a dose-response manner.
The non-specific analyte, horse cytochrome C, was not
detected in the parallel assay.

| Analyte | | |
|---|---|---|
| Concentration (ng/mL) | Fc Fragment (OD) | Horse Cytochrome C (OD) |
| 500 | 2.2 | 0.3 |
| 250 | 2.1 | 0.2 |
| 125 | 1.6 | 0.1 |
| 62.5 | 0.9 | 0.1 |
| 31.25 | 0.6 | 0.15 |
| 15.625 | 0.2 | 0.1 |
| 0 | 0.1 | 0.15 |

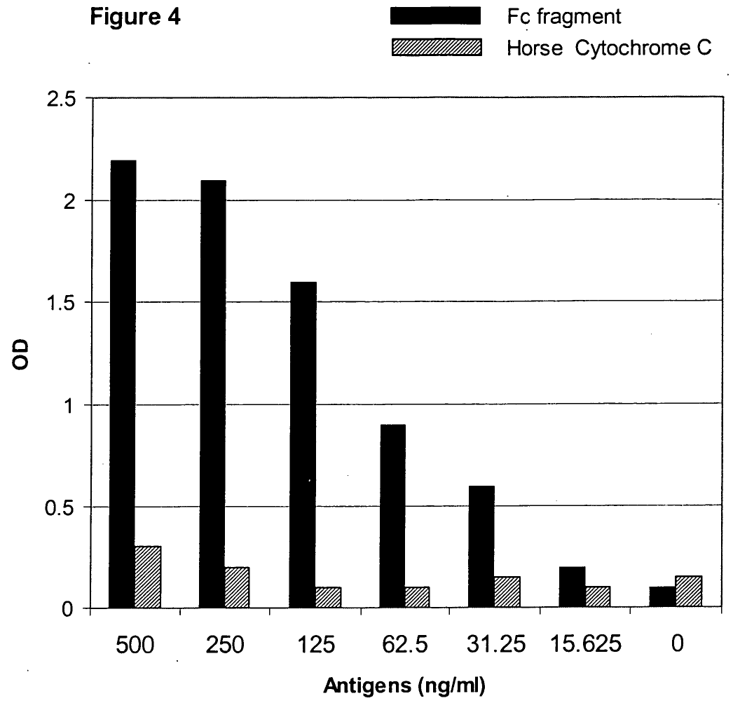

When graphed (FIG. 4), the results of this experiment unambiguously showed a dose response of the Fc fragment analyte, indicating that the detected signal was indeed due to specific capture of the analyte by the immobilized single capture antibody. In a parallel experiment, the same analyte titrations in the absence of immobilized antibodies failed to generate detectable signal (data not shown). Similarly, substitution with horse cytochrome C as an antigen failed to generate any single capture affinity signal using anti-Fc antibodies (Table 6 and FIG. 4), thus confirming the antigen specificity of the dose response. In this experiment, the upper end of the signal with the Fc fragment was saturated above 250 ng/ml concentration, and the signal faded into the background level (i.e. no analyte) below 15.625 ng/ml. Taken together, these results clearly demonstrated analyte-specific dose response, and confirmed the feasibility of using single capture affinity assay for protein quantification.

The invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

All references cited above are incorporated herein by reference in their entirety and for all purposes to the same extent as if each individual publication, patent or patent application was specifically and individually indicated to be incorporated by reference in its entirety for all purposes. Citation of a reference herein shall not be construed as an admission that such is prior art to the present invention.

1. A method for detecting an analyte in a sample comprising:
reacting an analyte with an affinity capture agent located at discrete areas of a solid support to bind the analyte to an analyte binding region of the affinity capture agent to form a complex analyte and the affinity of the capture agent;
reacting the complex with a chemical modifier capable of modifying amino acids of the affinity capture agent, wherein amino acids outside the analyte binding region are modified and the presence of bound analyte in the complex prevents modification at the analyte binding region;
dissociating the analyte from the complex to yield unmodified amino acids at the analyte binding region of the affinity capture agent;
reacting the unmodified amino acids with a signal generating component to yield a detectable signal; and
correlating the signal to detection of the analyte in the sample.

2. The method of claim 1 wherein the step of correlating the signal to the detection of analyte in the sample comprises using the detectable signal to quantitatively measure the analyte.

3. The method of claim 1 wherein the step of reacting the amino acids with the signal generating component is comprised of reacting the unmodified amino acids with a linking agent that does not bind the modified amino acids.

4. The method of claim 3 further comprising the step of reacting the linking agent with a signal tag that yields the detectable signal.

5. The method of claim 1 wherein the step of reacting the analyte with the affinity capture agent is comprised of reacting a polypeptide analyte with an antibody.

6. The method of claim 5 wherein the antibody is monoclonal.

7. The method of claim 1 wherein the amino acids are lysine.

8. The method of claim 1 wherein the analyte is a phosphorylated protein and the affinity capture agent is an antibody specific for the phosphorylated protein.

9. The method of claim 1 wherein the analyte is a blood pathogen and the affinity capture agent is an antibody specific for the pathogen.

10. The method of claim 9 wherein the pathogen is present in a biological sample.

11. The method of claim 1 wherein the analyte is a toxin and the affinity capture agent is an antibody specific for the toxin.

12. A kit for quantitatively detecting an analyte comprising:
an affinity capture agent located at discrete areas of a solid support wherein the affinity capture agent is comprised of amino acids susceptible of chemical modification and wherein the amino acids are present in an analyte binding area of the affinity capture agent,
a chemical modifier that reacts with the moiety;
a signal generating component comprising a linking agent capable of binding to the moiety, but not the modified amino acids; and
a signal tag that reacts with the linking agent to produce a detectable signal.

13. The kit of claim 12 wherein the affinity capture agent is an antibody.

14. The kit of claim 13 wherein the antibody is monoclonal.

15. The kit of claim 12 wherein the moiety is an amino acid is present in a non-analyte binding area of the affinity capture agent.

16. The kit of claim 13 wherein the affinity capture agent is disposed in an array.

17. The kit of claim 14 wherein the affinity capture agent is a monoclonal antibody specific for a phosphylated protein.

18. The kit of claim 14 wherein the affinity capture agent is a monoclonal antibody specific for a blood pathogen.

19. The kit of claim 14 wherein the affinity capture agent is a monoclonal antibody specific for a toxin.
